(19) United States
(12) Patent Application Publication
(10) Pub. No.: US 2025/0032800 A1
(43) Pub. Date: Jan. 30, 2025

(54) DEVICE, SYSTEM, AND METHOD FOR NEUROMODULATION

(71) Applicants: Arizona Board of Regents on behalf of Arizona State University, Scottsdale, AZ (US); Mayo Foundation for Medical Education and Research, Rochester, MN (US)

(72) Inventors: Derek Smetanick, Glendale, AZ (US); Shaun Victor, Chandler, AZ (US); Ashley Tse, Phoenix, AZ (US); Dhrasti Dalal, Phoenix, AZ (US); Alan Ramsey, Peoria, AZ (US); David Lott, Pheonix, AZ (US); Juergen Neubauer, Scottsdale, AZ (US); Jitendran Muthuswamy, Chandler, AZ (US)

(21) Appl. No.: 18/784,215

(22) Filed: Jul. 25, 2024

Related U.S. Application Data
(60) Provisional application No. 63/515,915, filed on Jul. 27, 2023.

(51) Int. Cl. A61N 1/36; A61N 1/02; A61N 1/05; A61N 1/378

(52) U.S. Cl. CPC A61N 1/36185; A61N 1/025; A61N 1/0556; A61N 1/3606; A61N 1/3787

(57) ABSTRACT

A neuromodulation device is provided that comprises a flexible layer comprising a non-conductive material, in which the layer is positioned in at least a partially tubular shape. The neuromodulation device comprises at least one cathodic band comprising at least one conductive cathodic contact housed by the layer. The neuromodulation device comprises an anodic band comprising at least one anodic contact housed by the layer. The neuromodulation device comprises is configured to receive a wireless signal to power and activate the at least one cathodic contact and the at least one anodic contact in a coordinated reset cycle.

### CROSS-REFERENCE TO RELATED APPLICATIONS

This application claims priority to U.S. Provisional Patent Application No. 63/515,915, filed on Jul. 27, 2023, incorporated herein by reference in its entirety.

### BACKGROUND OF THE INVENTION

Some neurological disorders may be conventionally treated using Botox injections, vibrotactile devices, or oral medications. However, these solutions do not always provide long lasting effects. Coordinated reset is a model that may have applications in treating many different neurological conditions. By taking advantage of neuroplasticity, coordinated reset desynchronizes neurons by resetting the phase and changing connection strengths of neurons. This method may be used to treat neurological conditions such as epileptic seizures, Parkinson's Disease, and Tourette's syndrome, and Spasmodic Dysphonia. Therefore, there is a need in the art for an implantable device capable of performing a coordinated reset of neurons. The present invention satisfies this need.

### SUMMARY OF THE INVENTION

Some embodiments of the invention disclosed herein are set forth below, and any combination of these embodiments (or portions thereof) may be made to define another embodiment.

In accordance with some implementations, there is neuromodulation device comprising a base layer, a first band for cathodic connections, and a second band for anodic connections. Each of the bands have at least one contact. The base layer houses the first and second band. Each of the bands may have two, three, or four contacts. The base layer may house two anodic bands and one cathodic band whereby the catholic band is positioned between the two anodic bands. The base layer may have a Young's Modulus no greater than 10 GPa. Each of the contacts may comprise platinum iridium. In some implementations, the one or more contacts of the respective first and second bands comprise a material that is corrosion resistant and biocompatible.

In accordance with some implementations, there is a neuromodulation system that may have a generator, a switch, a controller, and a housing. The generator is configured to generate a signal. The switch may be communicatively coupled or operatively coupled to the generator to receive the generated signal. The controller comprises processor and memory storing instructions that, when executed by the processor, cause the processor to perform operations, wherein the controller is communicatively coupled or operatively coupled to the switch. The operations of the controller may include providing input to the switch such that the signal from the generator energizes respective contacts of the first and second anode. The housing may be folded into a cuff around the nerve, wherein the housing includes a cathode; a first anode; and a second anode. In some implementations, the cathode is positioned between the first anode and the second anode, wherein each of the cathode, the first anode, and the second anode may have a contact respectively, wherein each of the respective contacts may be positioned to provide the signal to the nerve. In some implementations, both the first and second anode may comprise a second contact, respectively. In some implementations, the operations of the controller further comprise providing second input to the switch such that the signal from the generator energizes the second respective contacts of the first and second anode. In some implementations, the first and second anode may comprise a third contact, respectively. In some implementations, the operations of the controller further include providing a third input to the switch such that the signal from the generator energizes the third respective contacts of the first and second anode. In some implementations, the signal may be shaped to provide a reset, wherein the operations of the controller may further include repeating providing input, second input, and third input to provide a coordinated reset with the shaped signal.

In accordance with some implementations, a method for desynchronizing neurons by applying stimulus to a nerve is performed. The method including securing a housing to the patient, wherein securing may include wrapping the housing around the nerve, wherein securing may include ensuring that respective contacts from an anode and a cathode are sufficiently applied to a surface of the nerve to apply the stimulus; powering on a generator with a signal, wherein the generator is operatively coupled to the respective contacts; and providing desynchronization for the neurons by energizing respective contacts. In some implementations, the anode and the cathode further may have second respective contacts, wherein the method may include providing a second desynchronization for the neurons by energizing the second respective contacts. In some implementations, the anode and the cathode further have third respective contacts, wherein the method further comprises: providing a third desynchronization for the neurons by energizing third respective contacts. In some implementations, the method further comprises repeating each of the aforementioned desynchronizations until there is sufficient stimulation, and based on sufficient stimulation, the stimulation is terminated. In some implementations, the terminating stimulation is between the range of 20 to 45 minutes.

In accordance with some implementations, a neuromodulation device is provided. The neuromodulation device comprises a flexible layer comprising a non-conductive material, in which the layer is positioned in at least a partially tubular shape. The neuromodulation device comprises at least one cathodic band comprising at least one conductive cathodic contact housed by the layer. The neuromodulation device comprises an anodic band comprising at least one anodic contact housed by the layer. The neuromodulation device comprises is configured to receive a wireless signal to power and activate the at least one cathodic contact and the at least one anodic contact in a coordinated reset cycle.

In accordance with some implementations, a neuromodulation method of stimulating a nerve is provided. The method comprises positioning a neuromodulation device at least partially around a nerve. The device comprises a flexible layer comprising a non-conductive material. The device comprises at least one cathodic band comprising at least one conductive cathodic contact housed by the layer. The device comprises an anodic band comprising at least one anodic contact housed by the layer. The at least one cathodic contact and the at least one anodic contact interface with the nerve when the neuromodulation device is positioned at least partially around the nerve. The method comprises fastening neuromodulation device in position at least partially around the nerve by suturing sides of the layer to each other. The method comprises providing power to activate the at least one cathodic contact and the at least one anodic contact. The activated at least one cathodic contact and at least one anodic contact stimulate the nerve in a coordinated reset cycle.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements. The drawings are not to scale and are intended for use in conjunction with the explanations in the following detailed description.

FIGS. 1A through 1C depict an exemplary electrode patch device according to aspects of the present invention. FIG. 1A is a view of the exemplary electrode patch that is unfolded showing contacts. FIG. 1B depicts an isometric view of the exemplary electrode patch in a folded configuration showing cathodic and anodic bands. FIG. 1C is an alternative isometric view of patch capable of being sutured around a nerve as a cuff.

FIG. 2A to 2D are views of a system incorporating the exemplary electrode patch device of FIGS. 1A-1C. FIG. 2A outlines exemplary physical components. FIG. 2B is an electrical schematic of the pulse generator being directed by a switch. FIG. 2C is an exemplary switch as a MUX with an exemplary computing device. FIG. 2D is an exemplary switch as a MUX with exemplary connections.

FIGS. 3A through 3B depict the exemplary electrode patch device according to aspects of the present invention configured to deliver pulses to each of the rows of the electrodes.

FIG. 4 depicts a flowchart for an exemplary method of stimulating the nerve.

FIGS. 5A-G depict results of experiments. FIG. 5A depicts the exemplary cuff sized to be applied to the laryngeal nerve. FIG. 5B depicts a cutting apparatus. FIG. 5C depicts silicon tubing for the cuff being pinned down. FIG. 5D depicts impedance versus frequency for the cuff. FIG. 5E depicts a 3-cell configuration set up. FIG. 5F depicts cyclic voltammetry for material characterization. FIG. 5G depicts a Design of Experiments with varied voltage and frequency.

### DETAILED DESCRIPTION OF THE INVENTION

It is to be understood that the figures and descriptions of the present invention have been simplified to illustrate elements that are relevant for a clearer comprehension of the present invention, while eliminating, for the purpose of clarity, many other elements found in implantable devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass the specified value and/or variations of ±20%, ±10%, ±5%, ±1%, and ±0.1% from the specified value, as such variations are appropriate.

Various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Certain terminology is used in the following description for convenience only and is not limiting. For example, the words “right”, “left”, “lower,” “upper,” “back,” and “front” may designate components attached to the elongated member but are not limiting in any way on how the insert may be applied to the patient.

The terms “patient,” “subject,” “individual,” and the like are used interchangeably herein, and refer to any animal amenable to the systems, devices, and methods described herein. The patient, subject or individual may be a mammal, for example, a human.

Referring now in detail to the drawings, in which like reference numerals indicate like parts or elements throughout the several views, in various embodiments, presented herein are devices, systems, and methods for neuromodulation.

Spasmodic dysphonia (SD) is a type of neurological disorder that causes laryngeal muscle spasms, which may cause irregular speech in patients. In healthy conditions, normal processing of the sensory input is supposed to suppress sensory information during voice production, and not to interrupt voice. SD symptoms may be caused by abnormal processing of somatosensory feedback due to a pathological neural state of the neural network of involved cortex regions. In SD, the sensory input during voice production causes control signals to be sent to the larynx to close or open the larynx, similar to breathing or swallowing or airway protection. During speech planning, a couple of milliseconds before speech production, there are abnormal activities in certain cortex regions. Similar to other dystonias, SD is postulated to occur due to neuron synchronization.

The primary current treatment is to inject Botox into the abductor and/or abductor laryngeal muscles in order to immobilize the muscle, thus stopping spasms. However, this solution requires frequent retreatment and is only fully effective for a small portion of the treatment period. As such, there is a clinical need for a long-term solution to manage SD.

As contemplated herein, the present invention relates in part to an implantable electrode configured to engage with one or more nerves of a subject. For example, and without limitation, the invention relates to an electrode set in a cuff designed for the laryngeal nerve to treat SD. The device utilizes a coordinated reset to desynchronize neurons by phase resetting neurons and changing their connection strengths. For instance, the embodiments described herein relate to performing a coordinated reset to desynchronize the synchronized states of the sensorimotor cortex involved in voice production. In some embodiments, the device comprises a base layer, a first band for cathodic connections having at least one contact, and a second band for anodic connections having at least one contact, where the base layer houses the first band and the second band. For example, the electrode includes three conductive bands, each including three metal patches, housed in a silicone casing to minimize abrasion of the nerve.

### Electrode Patch Device

### Dimensions

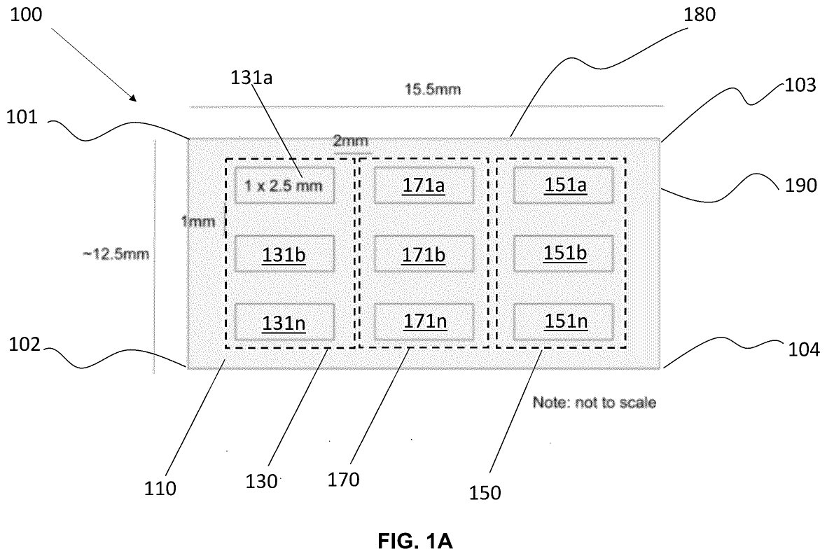

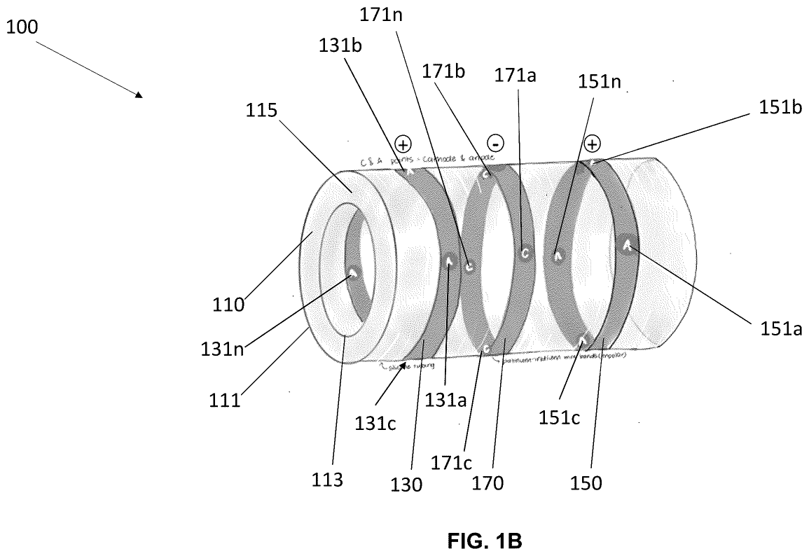

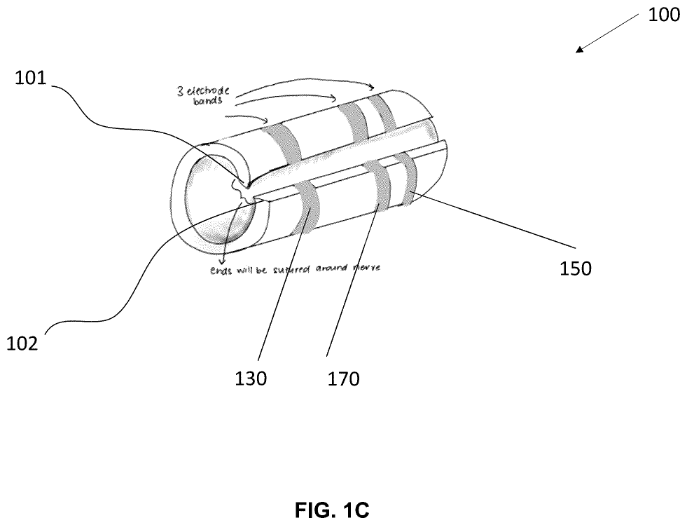

Referring now to FIG. 1A, shown is an exemplary electrode patch 100. The electrode patch 100 has a length 180 and width 190. The width 190 of the electrode patch 100 may be sized to wrap around the nerve of the patient to form the cuff as seen in FIG. 1C. The length 180 of the electrode patch 100 may be sized to extend along the length of the nerve. For example, the length 180 of the electrode patch 100 may be formed in a variety of lengths corresponding to an area to cover the nerve with one or more bands, such as bands 130, 150, and 170. Electrode patch 100 includes a base layer 110. One or more bands 130, 150, 170 may be coupled to the base layer 110. In some embodiments, the base layer 110 may house one or more components of the electrode patch 100. For example, the base layer 110 may house contacts with bands, such as contacts 171a, 171b . . . 171n of band 170. In another example, the base layer 110 may house contacts without bands. In some embodiments, the contacts may be embedded into the housing (i.e., the base layer 110). For example, the contacts may be embedded into the housing, such that the contacts are flush with the surface of the housing. In another example, the contacts may be embedded into the housing, such that a portion of the contacts protrude from the surface of the housing. In some embodiments, the contacts may be embedded into the housing such that the housing encases the contacts. As such, by encasing the contacts, the housing minimizes abrasion of the nerve. In some embodiments, the electrode patch 100 does not include bands 130, 150, 170, but rather, the contacts (e.g., contacts 131a, 131b . . . 131n, contacts 151a, 151b . . . 151n, and contacts 171a, 171b . . . 171n) are encased and arranged within the base layer 110

In some embodiments, the base layer 110 may be flexible, elastic, and/or rigid in parts. For example, certain regions of the base layer 110 may be flexible while other regions of the base layer 110 may be rigid. One or more contacts may be applied to the flexible regions of the base layer 110, thereby allowing the contacts to directly contact the surface of the nerve. In some embodiments, the base layer 110 may be flexible and elastic. In some embodiments, the whole of the base layer 110 may be flexible, elastic, or rigid. For example, the entirety of the base layer 110 may be flexible, such that the base layer 110 may wrap entirely around the surface of the nerve as a cuff as seen in FIGS. 1B and 1C. For example, corners 101 and 102 of the flexible base layer 110 may be sutured together, as seen in FIG. 1C. In another example, the sides of the base layer 110 that define the length 180 may be sutured to one another. In other embodiments, in an unfolded configuration, the base layer 110 may be shaped as an oval or another shape, as opposed to a rectangle, of sufficient length and/or width whereby, when configured in a cuff configuration, the base layer 110 may be wrapped and secured around the nerve or surrounding tissue, for example, via one or more sutures. In various embodiments, base layer 110 may have a series of premade apertures for sutures to attach thereto and anchor to the surrounding tissue. The apertures allow a surgeon to feed through a suture. Base layer 110 may be made up of biocompatible materials and/or nonconductive materials. The surface of base layer 110 may be textured so as to allow stability on the surrounding tissue. The surface of the base layer 110 may be smooth on one or both sides to prevent irritation to surrounding bodies, such as sensitive nerves. For instance, the base layer 110 may be formed of silicone to minimize abrasion of the nerve.

Referring now to FIG. 1B, shown is the exemplary electrode patch 100 formed in a cuff configuration with a side view of base layer 110. In a cuff configuration, the electrode patch 100 may be formed in a tubular shape having a hollow body. A sidewall 115 of the base layer 110 may define the thickness of the base layer 110. When the electrode patch 100 is wrapped around a body, such as a nerve, the electrode patch 100 is configured in the tubular shape having an inner diameter 113 and outer diameter 111. In one embodiment, the inner diameter 113 is sized to wrap around a body, such as the nerve of the patient, to provide electrical stimulation, such as, for neural desynchronization. In an alternative embodiment, the inner diameter 113 is sized to wrap around the nerve in part while leaving some gap between the inner surface of the base layer 110 and surface of the nerve. In an alternative embodiment, the inner diameter 113 may be sufficiently sized to partially wrap around the nerve in a circular, semi-circular, or partial circular fashion, such that the ends of the base layer 110 do not overlap one another, for example, as illustrated in FIG. 1C.

In various embodiments, the nerve may be the superior laryngeal nerve of the patient. The superior laryngeal nerve includes two branches, which are the external laryngeal nerve and the internal laryngeal nerve. The internal branch of the superior laryngeal nerve may be about 2.1±0.2 mm in diameter with the inner diameter 113 sufficiently fit against the outer diameter of the internal branch of the superior laryngeal nerve of the patient. For example, the inner diameter 113 of the electrode patch 100 may be about 2.0 mm to 2.5 mm for the internal branch of the superior laryngeal nerve but may be no smaller than the diameter of the internal branch. The thickness of the sidewall 115 of the base layer 110 may be about 0.2 mm to 0.5 mm. The outer diameter 111 may be about 3.5 to 4.0 mm. The inner 113 and outer 111 diameter may be sized together with the proper sidewall 115 such that base layer 110 may be sufficiently flexible. For example, the inner diameter 113 may be about 2.0 mm with the outer diameter 111 being about 4.0 mm. In the case where diameters of any nerve are larger, the electrode patch 100 need not wrap completely around the nerve in the cuff configuration. Instead, the electrode patch 100 may be partially open in the cuff configuration, as seen in FIG. 1C, and held with sutures.

In some embodiments, the electrode patch 100 may be positioned along either the right superior laryngeal nerve or the left superior laryngeal nerve. In other embodiments, one electrode patch 100 may be positioned along the right superior laryngeal nerve, and another electrode patch 100 may be positioned along the left superior laryngeal nerve. As such, a contralateral or bilateral placement of the electrode patch 100 may be implemented based on a severity of a disease and/or recommendation of the physician. Further, it is noted that the examples discussed herein relate to applying the electrode patch 100 along the internal branch of the superior laryngeal nerve, but it should be understood that the electrode patch 100 may be wrapped around other portions of the laryngeal nerve (e.g., the external branch of the laryngeal nerve) or around other nerves to stimulate the respective nerve.

In various embodiments, a width 190 of the electrode patch 100 in an unfolded configuration may be sized in such a way that the outer diameter 111 substantially matches the diameter of the applied nerve in order to be sufficiently tight around the nerve in the cuff configuration. In one embodiment, width 190 may be about 12.5 mm or sized such that an outer diameter 111 of the electrode patch 100 substantially matches the diameter of the applied superior internal laryngeal nerve.

In various embodiments, the base layer 110 may have a uniform thickness. In some embodiments, the base layer 110 may include a gradation of thicknesses. For example, base layer 110 may be thinner along or around the corners 101, 102, 103, and 104 or edges 180 and 190 while being thicker in the center.

Referring back to FIG. 1A, the length 180 of the electrode patch 100 in a folded/cuff configuration may be about 12.5 mm to 30 mm. The internal branch of the superior internal laryngeal nerve is typically 57.2±7.7 mm. In one embodiment, the electrode patch 100 may be applied to the laryngeal nerve and the length 180 of the electrode patch 100 may be about 23 to 30 mm. However, the electrode patch 100 need not extend the whole length of the laryngeal nerve. In alternative embodiment, the electrode patch 100 may have a length 180 of about 2.5 mm to 17.5 mm and placed on a portion of the laryngeal nerve.

### Bands and Contacts

In one embodiment, the electrode patch 100 has at least one cathodic band and at least one anodic band. In some embodiments, the bands may be embedded in the base layer 110. In other embodiments, the bands may be placed on a surface of the base layer 110, such that when the electrode patch 100 is formed in a folded/cuff configuration the bands face the interior of the tubular or partially tubular electrode patch 100. In a preferred embodiment, the electrode patch 100 is a tripolar patch which has two anodic bands 130 and 150 and one cathodic band 170. In some cases, the cathodic band 170 is preferably placed between both anodic bands 130 and 150. In other cases, the cathodic band 170 may be placed on either side of anodic bands 130 and 150. Each of the bands may have at least one contact. For example, cathodic band 170 may have any number of contacts, such as contacts 171a, 171b . . . 171n. Similarly, anodic band 150 may have any number of contacts, such as contacts 151a, 151b . . . 151n. Anodic band 130 may have any number of contacts, such as contacts 131a, 131b . . . 131n. For example, each band 130, 150, and 170 may have three or four contacts thereby having a total of 6 anodic contacts and 3 cathodic contacts or 8 anodic contacts and 4 cathodic contacts, respectively. Depending on the number of contacts, the size of contacts, the spacing of the contacts, and the like, the contacts are sufficiently spaced apart such that there is no electrical shorting when the electrode patch 100 is in unfolded configuration or cuff configuration. For example, when the electrode patch 100 is positioned in the cuff configuration around the nerve, the contacts may be sufficiently spaced apart from one another such that contacts do not touch as to prevent electrical shorting. For example, the contacts within a band may be spaced apart from one another so as to prevent electrical shorting within the band. Further, the contacts between bands may be spaced apart from one another so as to prevent electrical shorting across bands. In various embodiments, the contacts for the cathodic and anodic bands need not be sized the same. In various embodiments, the cathodic or anodic contacts may each have a length of about 2.5 mm, which may be substantially parallel to length 180, and may have a width of about 1.0 mm, which may be substantially parallel to width 190.

In various embodiments, the spacing between each contact within a band may be about 1.0 mm to 2.0 mm. In various embodiments, the widths of contacts within a band and the spacing between the contacts within the same band may be sized such that the contacts substantially fill the width 190 while leaving gaps at the edges of the electrode patch 100, in which the gaps range from about 0.5 to 0.75 mm. Similarly, in various embodiments, the length of the contacts and the spacing between the bands may be sized such that the contacts substantially fill the length 180 while leaving gaps at the edges of the electrode patch 100, in which the gaps range from about 0.5 mm to 0.75 mm.

In various embodiments, the width 180 of the electrode patch 100 may be about 23 to 30 mm. As such, the contacts may be sized accordingly. By way of example, a contact may be about 1.0 mm by 5.0 mm.

In various embodiments, the contacts of the cathodic band 170, which have a negative charge, and the contacts of the anodic bands 130 and 150, which have positives charges, may have two contacts per band instead of three per band. For example, anodic bands 130 and 150 may each have two contacts 131a and 131b and 151a and 151b, respectively. By way of another example, anodic bands 130 and 150 may each have three contacts 131a, 131b, and 131c and 151a, 151b, and 151c, respectively.

In various embodiments, the bands may be in a preferable tripolar configuration with the cathodic band 170 positioned between both anodic bands 130 and 150. For example, dimensions of the base layer 110 may include a width 190 of about 6 mm and a length 180 of about 23 mm with contacts about 2×5 mm with each band, such as bands 130, 150, and 170, having two contacts.

In various embodiments, cathodic band 170 and anodic bands 130 and 150 may each have three contacts. In a preferred embodiment, the three contacts per band may be arranged in a tripolar configuration. By way of example for a tripolar three contact design, the base layer 110 may have a width 190 of about 9.5 mm and a length 180 of about 23 mm, and the contacts may each have a size of about 2×5 mm or about 1×2.5 mm.

In some embodiments, the contacts may be activated in a coordinated pattern to stimulate the nerve coupled to the electrode patch 100. By activating contacts in a coordinated pattern, the electrode patch 100 is utilized to perform a coordinated reset. A cycle of a coordinated reset may include activating one or more contacts in a pattern. In some embodiments, the electrode patch 100 may repeat the coordinated reset pattern for a predetermined period of time to provide stimulation. A coordinated reset cycle may include, for example, but not limited to, activating contacts 131a, 171a, and 151a, either simultaneously or sequentially, then activating contacts 131c, 171c, and 151c, either simultaneously or sequentially, then activating contacts 131n, 171n, and 151n, either simultaneously or sequentially, and then contacts 131b, 171b, and 151b, either simultaneously or sequentially. The coordinated reset cycle may repeat and provide stimulation for a period of time. Another example coordinated reset cycle may include activating all contacts in band 130, then activating all contacts in band 150, and then activating all contacts in band 170. It is noted that the electrode patch 100 may utilize other coordinated reset cycle patterns by activating one or more contacts in various sequences. In some embodiments, the same stimulation pattern may be used for each coordinated reset cycle. In other embodiments, the stimulation patterns may vary between each coordinated reset cycle.

In some embodiments, the contacts may be activated by the electrode patch 100 receiving a wireless signal, converting the received signal to electrical power, and applying the power to the respective contacts. For example, the electrode patch 100 may receive high frequency power from a power generator (e.g., generator 205). For instance, the generator may provide an ultrasound signal via an ultrasound transmitter. The electrode patch 100 may include a transducer that receives the ultrasound signal. The electrode patch 100, via, for example, the transducer, may convert the ultrasound signal to direct current (DC) electrical power. The electrode patch 100 may provide the DC power to the respective contacts to stimulate the contacts. It is noted that electrode patch 100 may utilize other methods to power and activate the contacts. For example, the contacts may be activated via, but not limited to, optical powering, volume conductive high frequency currents, acoustic powering, passive coupling, and/or inductive coupling.

### Materials

Referring now to FIG. 1C, shown is an exemplary folded view of the electrode patch 100. In one embodiment, the electrode patch 100 may be sutured along edges in order for the electrode patch 100 to fit around the nerve. Given that the electrode patch 100 may be sutured around the nerve at its edges, a suture with sufficient strand strength should be chosen following a Knot breaking rule which says that about 70% of a strand's strength may be taken away. Strain on the suture should not exceed about 8% but should preferably be about or below 5%. In one embodiment, nylon with a size of 8-0 or 9-0 may be used as the suture. In an alternative embodiment, prolene with a size of 8-0 or 9-0 may be used as the suture. In an alternative embodiment, a silk suture may be used.

In order to prevent encapsulation of the electrode patch 100 by the body, inflammation, scarring, neural degeneration, and/or prevent abrasion to the nerve, the material of the electrode patch 100 fit around the nerve should not be too stiff given that nervous tissues are typically on the order of 3.15 to 10 kPA for Young's Modulus. In one embodiment, silicon tubing may be provided which has a Young's Modulus from 1 to 10 GPa. In alternative embodiments, parylene C (PA), polyimide (PI), and PDMS may be used. In another embodiment, polyurethane may be used.

In one or more embodiments, the bands 130, 150, and 170 are formed of a nonconductive material, and the contacts of each band may be formed from a conductive material. In some embodiments, the contacts of one band may be made of the same conductive material. In other embodiments, the contacts of one band may be made of a combination of conductive materials. In a preferred embodiment, the contacts may be platinum iridium. In an alternative embodiment, the contacts may be platinum gold. In other embodiments, the bands may not include contacts but rather be made of a conductive material or a combination of conductive materials, such as platinum iridium. For example, the bands may be 90% Platinum and 10% Iridium.

### System

### Electrical Configuration

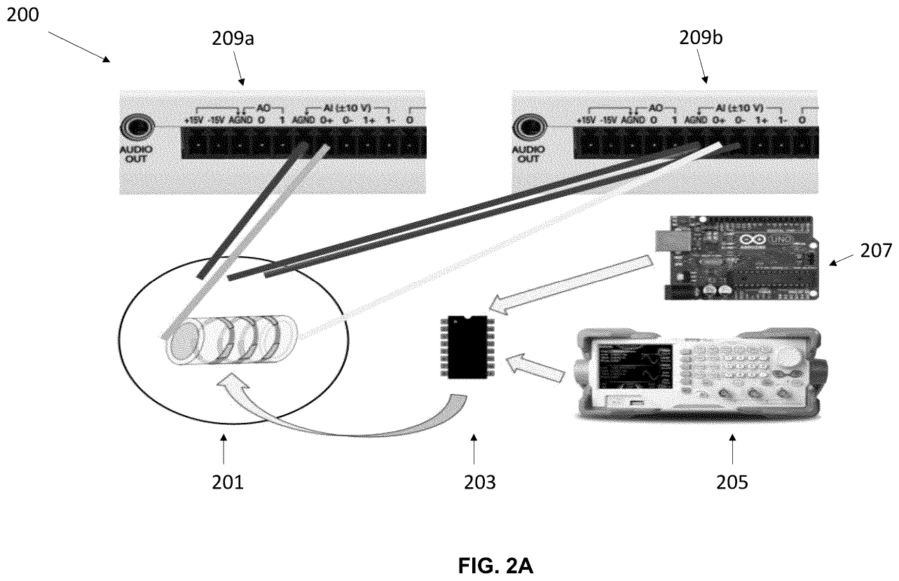

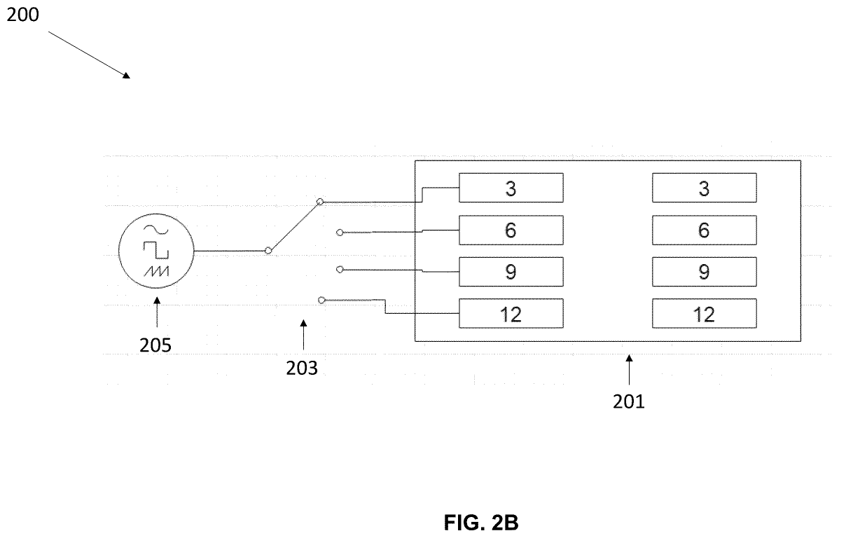

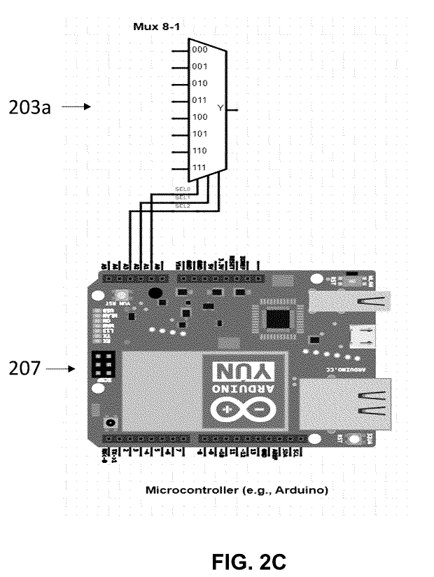

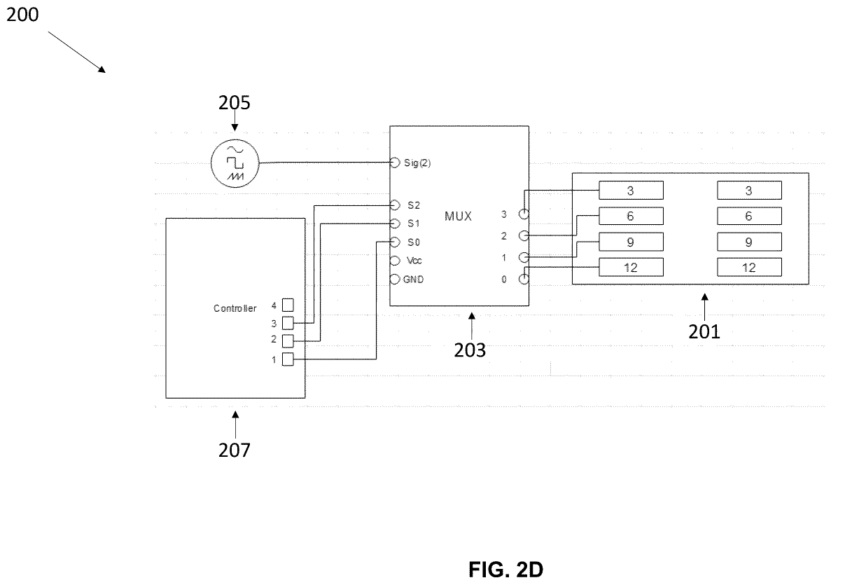

Referring now to FIG. 2A, shown is another aspect of the present invention that includes an exemplary system 200 for driving, powering, and/or grounding the electrode patch 201 (e.g., electrode patch 100). The system 200 includes the electrode patch 201; a switch 203; a generator 205 which may generate pulses; a microcontroller, controller, computer device, or any computing device 207; and power sources 209a and 209b with a ground, GND. For example, source 209a may be a myDAQ, whereby the source 209a may input a sinusoidal wave into a saline solution, and the power source 209b may also be a myDAQ that reads the respective signal.

Referring now to FIG. 2B, shown is an electrical schematic. Switch 203 may be any switch that connects to the electrode patch 201. In one embodiment, the switch 203 may comprise one or more MOSFETs, transistors, relays, or the like. It can be appreciated that the generator 205 may output monophasic, biphasic, square wave, sinewave, sawtooth wave, or any waveform or combination thereof capable of stimulating signals within the electrode patch 201 to provide a coordinated reset.

In a preferred embodiment, generator 205 outputs a biphasic square wave. Preferably, a delay between stimulation for each patch is set to about 80 ms. The stimulation period for each electrode patch is set to about 0.6 ms. Preferably, an ideal full coordinated reset cycle comprises five cycles—three cycles with the coordinated reset pattern (coordinated reset cycle on) and two cycles of coordinated reset being off (cessation). The period for a fully coordinated reset cycle (five cycles) is about 806 ms.

Referring now to FIG. 2C, the switch 203 may preferably be a multiplexer 203a. Multiplexers may include 2-1 with 1 select line; 4-1 with 2 select lines; 8-1 with 3 select lines; or 16-1 with 4 select lines based on the respective number of rows for a clock framework which is discussed further below. The multiplexers may include select lines which may be denoted by S0, S1, S2, and etc. whereby select lines select the corresponding output Y. For example, 8-1 MUX may be connected as represented in FIG. 2C to a controller 207 or any suitable computing device.

Referring now to FIG. 2D, with the appropriate signal provided by generator 205, S0, S1, and S2 are used to select the appropriate signal for the clock framework which is discussed in detail below. Within the clock model, a pause may be generated using hardware, software, or a combination of both. For example, the pause may be generated using computer code that is encoded into controller 207.

By way of example, the switch 203 may be a CD74HC4051-EP Analog Multiplexer and Demultiplexer by Texas Instruments, which, for the Multiplexer, has an 8-1 framework with three select lines S0, S1, and S2.

Connections from MUX/DEMUX 203 to 201 may preferable be stainless steel. Similarly, connections between MUX/DEMUX 203 to 205 and 203 to 207 may preferably be stainless steel. The stainless steel may be 0.05 mm in diameter. However, any wiring with biocompatible conductive material may be used.

### Clock Model

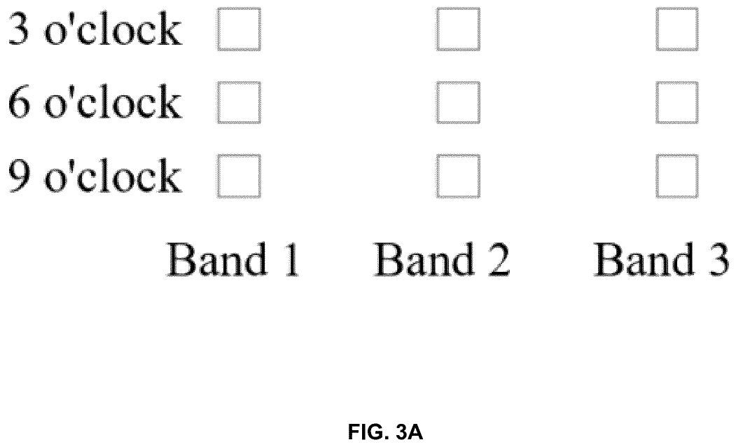

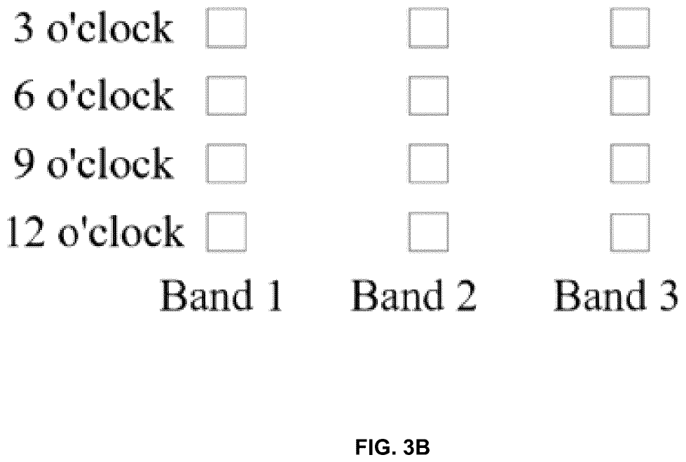

Referring now to exemplary FIGS. 3A and 3B, shown is the nonlimiting clock model that activates contact rows from top to bottom preferably with 3 rows and 4 rows respectively. For example, with respect to FIG. 3B, the 3 o'clock contact row may include contacts 131a, 171a, and 151a, illustrated in FIG. 1B, the 6 o'clock contact row may include contacts 131c, 171c, and 151c, the 9 o'clock contact row may include contacts 131n, 171n, and 151n, and the 12 o'clock contact row may include contacts 131b, 171b, and 151b. It can be appreciated that the hardware driving 3 or 4 rows, or any number of rows, may be altered with the appropriate hardware such as a MUX/DEMUX with S0 to SN select lines as needed. More or fewer rows may be alternatively added as needed based on the length 180 and/or width 190 dimensions described above in FIG. 1B.

In an alternative embodiment, the clock model may be reversed. For example, the clock model may move from 9, 6, to 3 o'clock. Alternatively, the clock model need not go in order from top to bottom or bottom to top. The clock model may hit every row within a period before returning to the starting position. Or any other pattern may be used to coordinate a reset.

Referring now back to FIG. 2B, generator 205 may preferably output a biphasic square wave which may be a pulse train. This biphasic square wave may have a cathodic phase followed by an anodic phase. For example, the cathodic pulse may have a high amplitude following a smaller cathodic amplitude pulse. The stimulating voltage may be preferably about 1.5V, a pulse width of about 1.5 ms, and a stimulation frequency of about 120 Hz. Alternatively, the stimulating voltage may range from about 1 to 1.75V and have a pulse duration of 0.15 ms while at 120 Hz. Alternatively, the stimulating voltage may range from about 0.5 to 2V, include a range of frequencies from about 60 Hz to 200 Hz, and/or include a range of pulse widths from about 0.1 ms to 1.5 ms. Electrode patch 201 may provide stimulation for about 30 minutes.

### Method

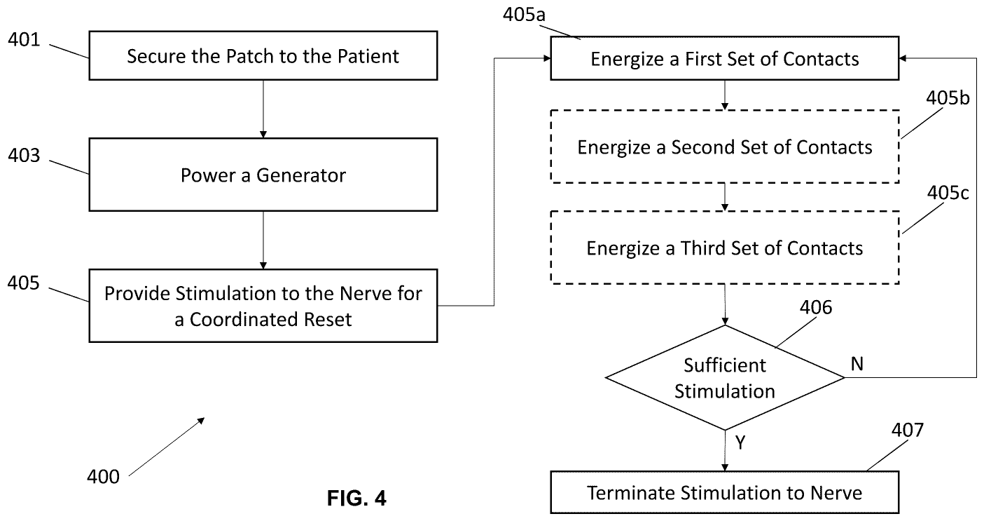

Referring now to FIG. 4, shown is exemplary method 400 for treating a neurological condition of a subject. Method 400 begins with step 401, which involves securing the patch (e.g., the electrode patch 100) to the patient's surrounding tissues and/or nerve. Examples include securing the patch to surrounding tissues or structures within the patient by way of anchoring the surrounding tissues or structures to corners, edges, or outer regions of the base layer 110 of the electrode patch 100. The patch includes the contacts, which are housed in the base layer 110 described herein. Securing the patch may further include folding the flexible or partially flexible base layer 110 partially around the nerve such that the housed contacts are positioned at, around, or on the nerve surface to provide stimulation.

By way of another example, the flexible or partially flexible base layer 110 of the electrode patch 100 may be positioned around the nerve in whole or in part such that the electrode patch 100 is folded into a cuff or semi-cuff around at least a portion of the nerve. As described herein, a surgeon may suture corners, edges, or outer regions of the base layer 110 to one another to secure the cuff or semi-cuff around the nerve such that the contacts are positioned, at, around, or on the nerve to provide stimulation.

In step 403, a generator (e.g., generator 205) is powered on. Generator 205 may preferably output a wireless signal to the patch to power the contacts. The wireless signal may be, for example, but not limited, to a biphasic square wave, which may be a pulse train. This biphasic square wave may have a cathodic phase followed by an anodic phase where the cathodic pulse may have a high amplitude following a smaller cathodic amplitude pulse. The stimulating voltage may be preferably about 1.5V, with a pulse width of about 1.5 ms and a stimulation frequency of about 120 Hz. Alternatively, the stimulating voltage may range from about 1V to 1.75V and have a pulse duration of 0.15 ms while at 120 Hz. Alternatively, the stimulating voltage may range from about 0.5V to 2V, with a range of frequencies from 60 Hz to 200 Hz, and/or a range of pulse widths from 0.1 ms to 1.5 ms.

In step 405, stimulation is provided to the nerve and includes step 405a that involves energizing at least a first set of contacts as described above. In an alternative embodiment, step 405 may further include steps 405b and/or 406c. Sets of energized contacts may be energized using the clock model or the period model described herein to desynchronize neurons. By way of example, steps 405a, 405b, and 405c may involve energizing the first row (3 o'clock), the second row (6 o'clock), and the third row (9 o'clock) of contacts, respectively, as illustrated in FIGS. 3A-B. Without limitation, method 400 may also include step 405d (not shown) to activate contacts in the 12 o'clock position. As shown, assessment of sufficient stimulation may include feedback from the patient or an expected time threshold such as 30 mins of activation. Step 405 and its sub-steps (e.g., 405a) may be performed in the exemplary clock model described herein until complete. For the cases in which stimulation is not sufficient (e.g., the coordinated reset is not complete) (step 406:N), the patch may activate the contacts in a coordinated reset cycle to provide further stimulation. For the cases in which stimulation is complete (step 406:Y), stimulation to the nerve is terminated at step 407. In some cases, terminating stimulation to the nerve may involve powering off the generator. In other cases, terminating stimulation to the nerve may involve powering off the contacts of the patch.

In some embodiments, a patient or another person (e.g., the patient's doctor) may activate and power the patch via a handheld device. As such, when the patient is experiencing symptoms of spasmodic dysphonia, the patient may treat their symptoms by powering the patch, which in turn provides stimulation to the nerve in a coordinated reset cycle.

### Experimental Examples

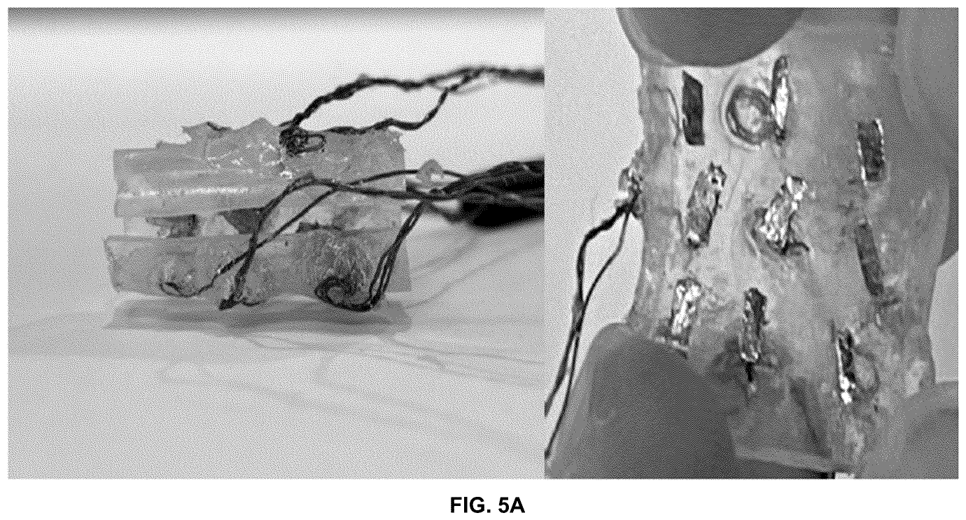

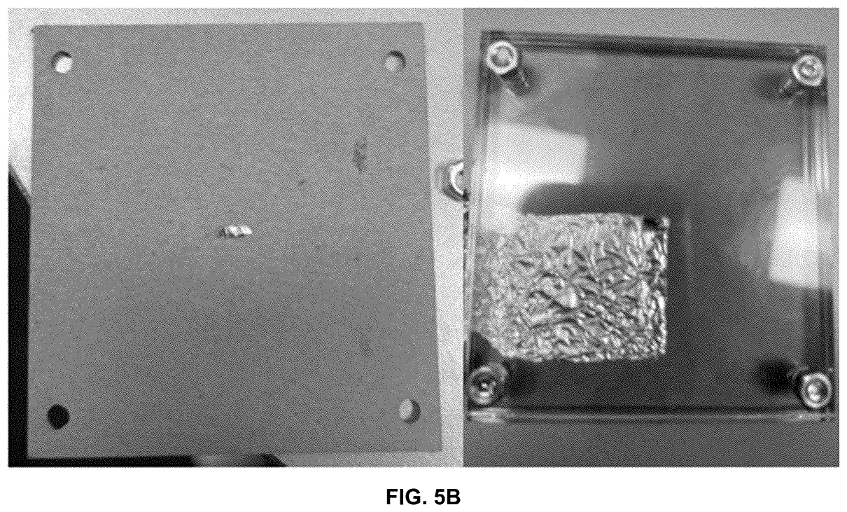

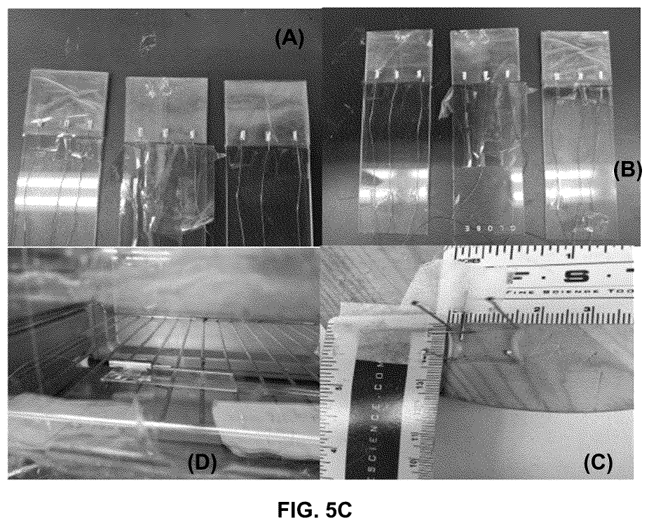

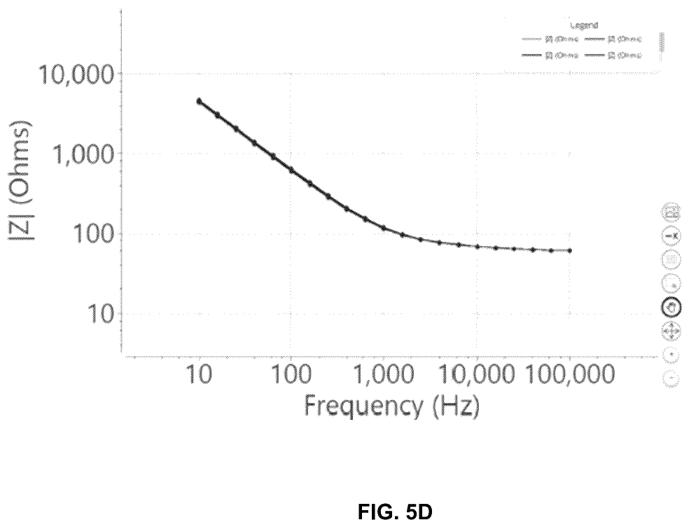

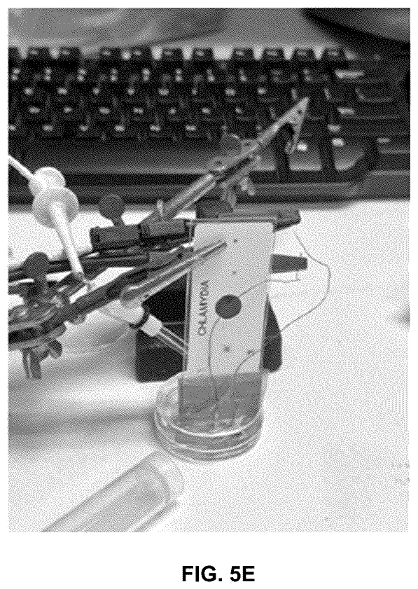

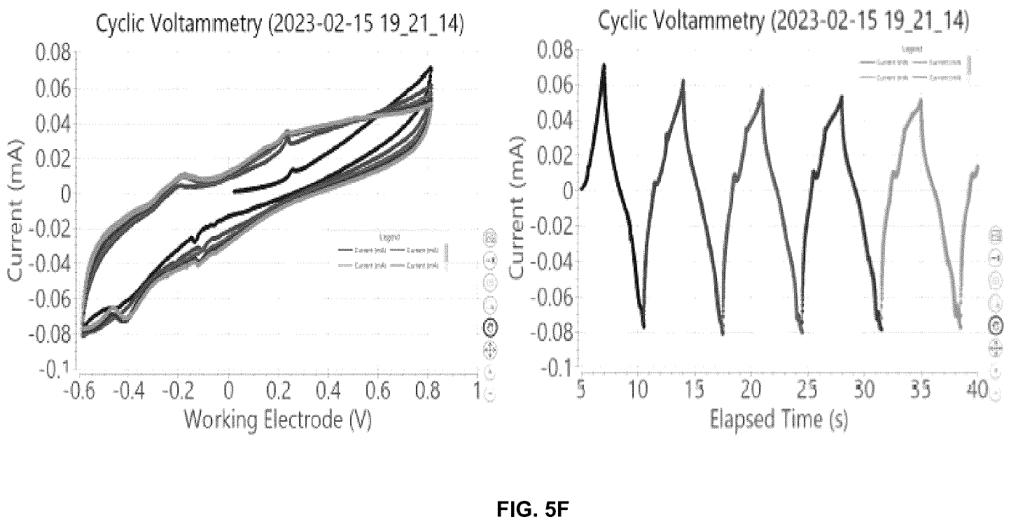

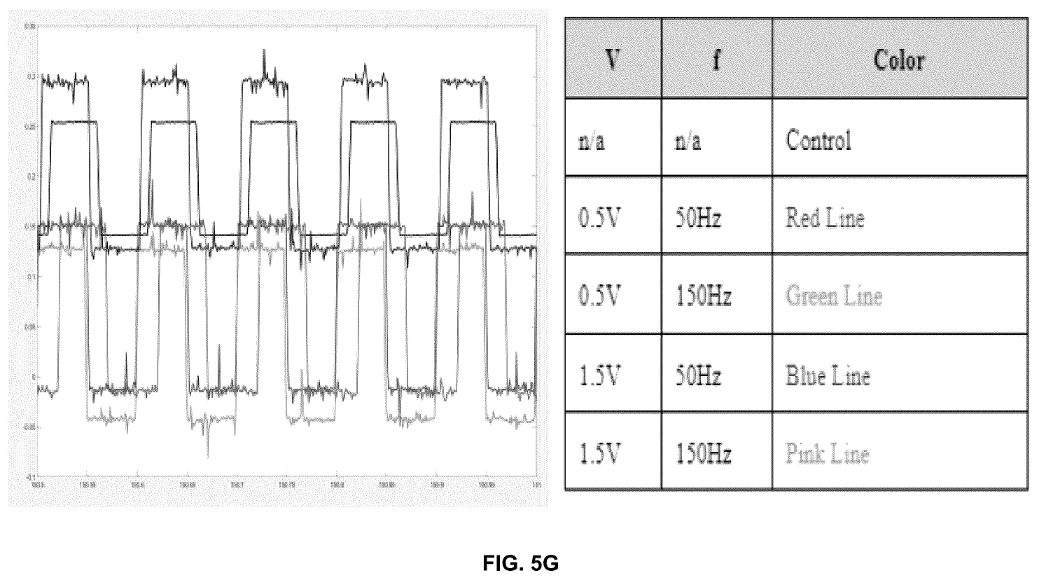

Referring now to FIG. 5A, shown on the left in FIG. 5A-1 is the completed cuff with nine secured contacts and shown on the right in FIG. 5A-2 is the opened-up cuff showing the 3×3 grid of platinum iridium contacts. Platinum iridium patches were cut from a platinum iridium sheet (0.01 inches×0.5 inches×0.866 inches) using an apparatus seen in FIG. 5B. Nine platinum iridium patches were used in the cuff electrode. A 17.5 mm long piece of silicone tubing was cut out and laid flat by pinning the tubing down with mini nails as seen in FIG. 5C on the bottom right side (C). While the cuff may preferably be an inner diameter of 2 mm and an outer diameter of 4 mm for the laryngeal nerve, a prototype of the silicon cuff created was 4 mm and 6 mm with an inner and outer diameter, respectively. Tweezers were used to make small incisions and thread the contacts through the cuff. A thick wire of three initial wires was made while still ensuring that our contacts remained level on the silicone tubing.

Referring now to FIG. 5C(a), (b), (c), and (d), stainless steel wires were attached to platinum iridium by coating the wires with silver epoxy. These were placed in an oven and baked for 10 minutes at 350 degrees. Silver epoxy was then recoated in the same area and baked again for an additional ten minutes to further strengthen. From there, marine epoxy was applied and baked at 350 degrees for five minutes. If more strength is needed, another layer of marine epoxy may be added as needed. These steps may be used regardless of the contact size.

After completing the prototype of the cuff electrode, jumper wires were soldered on to perform testing. A continuity test was conducted. Using a breadboard, testing was performed with an Arduino microcontroller with code that has the coordinated reset timing pattern.

Materials characterization with impedance spectroscopy and cyclic voltammetry (CV) was conducted. For impedance spectroscopy, a 2-cell and 3-cell configuration were created, and patches were confirmed to have a low impedance as desired. CV confirmed our contacts can take the theoretical voltage range of −0.6 to 0.8V. Shown in FIG. 5D are the results of the 2-cell configuration. Shown in FIG. 5E is the configuration for the 3-cell set up. Shown in FIG. 5F is current versus voltage graph for the cyclic voltammetry.

Referring now to FIG. 5G, a result of a Design of Experiments (DOE) is shown. The DoE was conducted with 4 experiments, altering the voltage and frequency of the biphasic pulse train going through the cuff electrode and a control voltage applied with 5V amplitude and 100 Hz frequency. Each trial was 5 minutes, and the data is a representative 0.5 second sample. For validation, an input signal was fed, and a resulting wave was measured. For a DOE, two parameters were selected with two levels each. The first one is input voltage, which was set to either 0.5V or 1.5V. The second one is the input frequency, which was set to either 50 Hz or 150 Hz. Prior to running the DOE, control tests were conducted where the cuff was placed in the saline solution, but not connected to the Arduino. A square wave was measured as the output when a sine wave was the input. The output was attenuated.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A neuromodulation device comprising:
a flexible layer comprising a non-conductive material, the layer being positioned in at least a partially tubular shape;
at least one cathodic band comprising at least one conductive cathodic contact housed by the layer; and
an anodic band comprising at least one anodic contact housed by the layer;
wherein the device is configured to receive a wireless signal to power and activate the at least one cathodic contact and the at least one anodic contact in a coordinated reset cycle.

2. The neuromodulation device of claim 1, wherein the layer houses the at least one cathodic contact and the at least one anodic contact such that a contact surface of the at least one cathodic contact and a contact surface of the at least one anodic contact are flush with a surface of the layer.

3. The neuromodulation device of claim 1, wherein the layer houses the at least one cathodic contact and the at least one anodic contact such that the at least one cathodic contact and the at least one anodic contact are completely embedded within the layer.

4. The neuromodulation device of claim 1, wherein the neuromodulation device comprises a length of about 12.5 millimeters (mm) to 30 mm.

5. The neuromodulation device of claim 1, wherein the layer comprises silicone.

6. The neuromodulation device of claim 1, wherein the layer, the at least one cathodic band, and the at least one anodic band are comprised of corrosion resistant and biocompatible materials.

7. The neuromodulation device of claim 1, wherein the at least one cathodic band and the at least one anodic band are comprised of a non-conductive material.

8. The neuromodulation device of claim 1, wherein, when positioned in a tubular shape, the layer comprises a diameter of about 2.0 millimeters (mm) to 2.5 mm.

9. The neuromodulation device of claim 1, wherein the at least one cathodic contact and the at least one anodic contact comprise platinum iridium.

10. The neuromodulation device of claim 1, wherein the at least one cathodic band comprises at least three cathodic contacts, and the at least one anodic band comprises at least three anodic contacts.

11. The neuromodulation device of claim 10, wherein the coordinated reset cycle comprises activating one of the at least three cathodic contacts and one of the at least three anodic contacts.

12. The neuromodulation device of claim 11, wherein the one cathodic contact and the one anodic contact are activated simultaneously or sequentially.

13. The neuromodulation device of claim 11, wherein a pattern of the coordinated reset cycle comprises activating another one of the at least three cathodic contacts and another one of the at least three anodic contacts.

14. The neuromodulation device of claim 1, wherein the at least one cathodic band comprises a first cathodic band and a second cathodic band disposed on opposite sides of the anodic band.

15. The neuromodulation device of claim 1, wherein the layer is positioned around a nerve, the layer having a Young's Modulus of 1 to 10 GPa.

16. A neuromodulation method of stimulating a nerve comprising:
positioning a neuromodulation device at least partially around a nerve, wherein the device comprises:
a flexible layer comprising a non-conductive material,
at least one cathodic band comprising at least one conductive cathodic contact housed by the layer, and
an anodic band comprising at least one anodic contact housed by the layer, wherein the at least one cathodic contact and the at least one anodic contact interface with the nerve when the neuromodulation device is positioned at least partially around the nerve,
fastening neuromodulation device in position at least partially around the nerve by suturing sides of the layer to each other; and
providing power to activate the at least one cathodic contact and the at least one anodic contact, wherein the activated at least one cathodic contact and at least one anodic contact stimulate the nerve in a coordinated reset cycle.

17. The neuromodulation method of claim 16, wherein the nerve comprises a superior laryngeal nerve.

18. The neuromodulation method of claim 16, wherein providing power to activate the at least one cathodic contact and the at least one anodic contact comprises transmitting a wireless signal to the neuromodulation device.

19. The neuromodulation method of claim 16, wherein the at least one cathodic band comprises at least three cathodic contacts, and the at least one anodic band comprises at least three anodic contacts, wherein stimulating the nerve in the coordinated reset cycle comprises activating one of the at least three cathodic contacts and one of the at least three anodic contacts.

20. The neuromodulation method of claim 19, wherein stimulating the nerve in the coordinated reset cycle comprises activating another one of the at least three cathodic contacts and another one of the at least three anodic contacts.
